(19) United States
(12) Patent Application Publication
(10) Pub. No.: US 2006/0276630 A1
(43) Pub. Date: Dec. 7, 2006

(54) Method for detecting protein crystal, apparatus for detecting protein crystal and program for detecting protein crystal

(75) Inventors: Hirofumi Matsuzaki, Fukuoka-shi (JP); Hideyuki Miyatake, Tatsuno-shi (JP); Seong Hoon Kim, Ibo-gun (JP); Kunio Miki, Takatsuki-shi (JP)

Correspondence Address: HAMRE, SCHUMANN, MUELLER & LARSON P.C., P.O. BOX 2902-0902, MINNEAPOLIS, MN, 55402, US

(73) Assignees: MATSUSHITA ELECTRIC INDUSTRIAL CO., LTD., Kadoma-shi (JP); RIKEN, Wako-shi (JP)

(21) Appl. No.: 10/554,579

(22) PCT Filed: Apr. 28, 2004
(86) PCT No.: PCT/JP04/06160

§ 371 Date: Oct. 27, 2005

(30) Foreign Application Priority Data
Apr. 28, 2003 (JP) 2003-123984

(51) Int. Cl. G06F 19/00; C07K 14/47

(52) U.S. Cl. 530/350; 702/19

(57) ABSTRACT

It is an object of the present invention to provide a protein crystal detection method, protein crystal detection apparatus, and protein crystal detection program with which protein crystals can be efficiently detected at a high reliability. To detect protein crystals with the present invention, an observed image obtained by observing a protein solution during crystallization by the vapor diffusion method is differentiated to give a differentiated image constituted by brightness change information indicating how much brightness has changed. Noise then is eliminated from this differentiated image, after which the differentiated image is binarized at a threshold for extracting a characterizing portion, which gives a binarized image in which the portion exhibiting a large change in brightness is left behind as the characterizing portion. Crystallization is evaluated by determining whether or not there are protein crystals from this characterizing portion of the binarized image. Thus, protein crystal detection, which up to now has relied exclusively on visual observation by observers, can be carried out efficiently with high reliability.

### TECHNICAL FIELD

The present invention relates to a protein crystal detection method, protein crystal detection apparatus, and protein crystal detection program with which the protein crystals in a protein solution held in a crystallization vessel are detected.

### BACKGROUND ART

There has been a growing trend in recent years toward effectively utilizing genetic information in the fields of medicine and so forth, with most of the work in terms of the basic technology going into analyzing the structure of proteins obtained as a result of gene expression. This structural analysis of proteins identifies the three-dimensional structure of proteins, and is performed by means of X-ray diffraction or another such method.

To analyze the structure of a protein by X-ray diffraction, it is required first to crystallize the protein to be analyzed, and one known method for this protein crystallization is vapor diffusion. With this method, crystals are produced gradually while the protein solution containing the protein to be crystallized is kept in a saturated state by absorbing the solvent component that evaporates from the protein solution with a crystallization solution contained in the same vessel. Special vessels and apparatus for performing the crystallization of proteins using this vapor diffusion method have been proposed in the past (see, for example, JP 2002-233702A and JP 2002-179500A).

However, the crystal growth conditions for promoting crystallization cannot be identified theoretically at the present time, so a screening method has to be used, in which the optimal conditions are found from the results of statistically executing numerous tests under various conditions. Consequently, up to now tests of protein solutions to be crystallized had to be conducted repeatedly under various crystal growth conditions, that is, various sets of conditions in which the type and concentration of crystallization solution and the growth temperature were varied.

Such tests have been conducted by putting a protein solution and a crystallization solution in a crystallization vessel, such as a microplate for crystallization, keeping this vessel in a thermostatic chamber set to a specific temperature, and observing whether or not crystallization occurs, and the extent thereof, over time. The observation work entailed by protein crystal detection has up to now relied exclusively on manual labor, in which the test personnel performed the work of recording data about the progress of crystallization by taking the crystallization vessel out of the thermostatic chamber and visually observing the protein solution under a microscope.

### DISCLOSURE OF INVENTION

However, such observation work involves visually discerning and detecting tiny crystals in a protein solution, which meant that accurately and efficiently detecting protein crystals has been difficult. For example, in an observed image taken of a protein solution, noise components, such as the silhouettes of droplets or shadows attributable to the shape of the crystallization vessel or to sediment and other such solid foreign matter, were present in addition to the protein crystals that are supposed to be detected, and it took considerable skill on the part of special test personnel to tell the difference between these noise components and the crystals that were to be detected. Consequently, there was inevitably inconsistency in work efficiency and reliability due to individual differences between testers, and the work efficiency and reliability of detection results in crystal detection work in the past have been less than satisfactory.

In view of this, it is an object of the present invention to provide a protein crystal detection method, protein crystal detection apparatus, and protein crystal detection program with which protein crystals can be efficiently detected at a high reliability.

To achieve the stated object, the protein crystal detection method of the present invention for detecting protein crystals comprises a differentiation step of observing a protein solution and differentiating the observed image, thereby producing a differentiated image constituted by brightness change information indicating how much brightness has changed, a binarization step of binarizing the differentiated image at a threshold for extracting a characterizing portion, thereby obtaining a binarized image in which the portion of the brightness change information exhibiting a large change in brightness is left behind as the characterizing portion, and a crystallization evaluation step of determining from the characterizing portion of the binarized image whether or not there are protein crystals and/or whether or not crystallization has proceeded.

Also, to achieve the stated object, the protein crystal detection apparatus of the present invention for detecting protein crystals comprises an observed image memory for storing observed images obtained by observing a protein solution, a differentiation processor for differentiating an observed image in the observed image memory and thereby producing a differentiated image constituted by brightness change information indicating how much brightness has changed, a binarization processor for binarizing the differentiated image produced by the differentiation processor at a threshold for extracting a characterizing portion, thereby obtaining a binarized image in which the portion of the brightness change information exhibiting a large change in brightness is left behind as the characterizing portion, a binarized image memory for storing binarized images obtained from the binarization processor, and a crystallization evaluator for determining from the characterizing portion of the binarized images in the binarized image memory whether or not there are protein crystals and/or whether or not crystallization has proceeded.

Further, to achieve the stated object, the protein crystal detection program of the present invention for executing on a computer processing for detecting protein crystals produced in a protein solution comprises a differentiation step of observing a protein solution and differentiating the observed image, thereby producing a differentiated image constituted by brightness change information indicating how much brightness has changed, a binarization step of binarizing the differentiated image at a threshold for extracting a characterizing portion, thereby obtaining a binarized image in which the portion of the brightness change information exhibiting a large change in brightness is left behind as the characterizing portion, and a crystallization evaluation step of determining from the characterizing portion of the binarized image whether or not there are protein crystals and/or whether or not crystallization has proceeded.

The work entailed by protein crystal detection has up to now relied exclusively on visual observation by observers, but with the present invention, this work can be automated and performed efficiently with high reliability. Also, the protein crystal detection method of the present invention can be used for the screening of protein crystallization conditions by vapor diffusion or another such method, and the protein crystal detection apparatus of the present invention can be used as a protein crystal manufacturing apparatus or an apparatus for screening protein crystallization conditions by vapor diffusion or another such method.

### BRIEF DESCRIPTION OF DRAWINGS

FIG. 1 is a perspective view of the protein crystal detection apparatus in an embodiment of the present invention;

FIG. 2 is a perspective view of the crystallization plate used in the protein crystal detection apparatus in an embodiment of the present invention;

FIGS. 3 and 5 are cross-sectional views of the portion of a crystallization plate used in the protein crystal detection method in an embodiment of the present invention;

FIG. 4 is a partial cross-sectional view of the observation component of the protein crystal detection apparatus in an embodiment of the present invention;

FIG. 6 is a block diagram illustrating the constitution of the control system of the protein crystal detection apparatus in an embodiment of the present invention;

FIG. 7 is a function block diagram illustrating the processing mechanisms of the protein crystal detection apparatus in an embodiment of the present invention;

FIG. 8 is a flowchart of the protein crystal detection method in an embodiment of the present invention;

FIGS. 9A and 9B are partial cross-sectional views of the crystallization plate used in the protein crystal detection method in an embodiment of the present invention;

FIG. 10 is a diagram of an observed image in the protein crystal detection method in an embodiment of the present invention;

FIG. 11 is a diagram of a differentiated image in the protein crystal detection method in an embodiment of the present invention;

FIG. 12 is a diagram of a differentiated image (masked) in the protein crystal detection method in an embodiment of the present invention;

FIG. 13 is a diagram of a line-thinned image in the protein crystal detection method in an embodiment of the present invention;

FIG. 14 is a diagram of a differentiated image (noise eliminated) in the protein crystal detection method in an embodiment of the present invention

FIG. 15 is a diagram of a binarized image in the protein crystal detection method in an embodiment of the present invention;

FIGS. 16A and 16B are diagrams illustrating the noise recognition processing in the protein crystal detection method in an embodiment of the present invention;

FIG. 17 is a diagram illustrating the noise recognition processing in the protein crystal detection method in an embodiment of the present invention; and

FIGS. 18A and 18B are diagrams illustrating the noise recognition processing in the protein crystal detection method in an embodiment of the present invention.

### DESCRIPTION OF THE INVENTION

The protein crystal detection method of the present invention preferably further comprises a vessel noise elimination step of eliminating at least one of brightness change information and a characterizing portion originating in the crystallization vessel that holds the protein solution to be crystallized, from at least one of the differentiated image and the binarized image.

In the protein crystal detection method of the present invention, it is preferable that the vessel noise elimination step involves eliminating at least one of brightness change information and a characterizing portion of a specific region on the basis of given information.

The protein crystal detection method of the present invention preferably further comprises a line-thinned image production step of obtaining a line-thinned image constituted by a plurality of lines by subjecting the differentiated image to line thinning after being binarized at a noise extraction threshold, a noise recognition step of detecting lines considered to be noise by individually recognizing the shapes of the plurality of lines included in the line-thinned image, and a noise elimination step of eliminating at least one of brightness change information and characterizing portion corresponding to the lines considered to be noise and detected in the noise recognition step, from at least one of the differentiated image and the binarized image.

In the protein crystal detection method of the present invention, it is preferable that the noise recognition step includes a step in which the length and number of branches of lines are found, and any lines whose length is over a predetermined specific value and whose number of branches is less than a predetermined value are considered to be noise.

In the protein crystal detection method of the present invention, it is preferable that the noise recognition step includes a step in which the length and linearity of lines are recognized, and any lines whose length is over a predetermined specific value and whose linearity is higher than a predetermined value are considered to be noise.

In the protein crystal detection method of the present invention, it is preferable that the noise recognition step includes a step in which the length of lines and the dispersion extent compared to the approximated line of the detected line are found, and any lines whose length is over a predetermined specific value and whose dispersion extent is less than a predetermined value are considered to be noise.

The protein crystal detection method of the present invention preferably further comprises an observation step of capturing with a camera an image of a protein solution held in a crystallization vessel, and a storage step of storing in an observed image memory the observed image captured by the camera, wherein the differentiation step is performed on images stored in the observed image memory.

The protein crystal detection method of the present invention also can be used as a method for detecting protein crystals generated in the screening of protein crystallization conditions. Other than the use of the detection method of the present invention, there are no particular restrictions on this screening method, and any conventional technique can be utilized.

Next, the protein crystal detection apparatus of the present invention preferably further comprises a vessel noise eliminator for eliminating at least one of brightness change information and a characterizing portion originating in the crystallization vessel that holds the protein solution to be crystallized, from at least one of the differentiated image in the differentiated image memory and the binarized image in the binarized image memory.

The protein crystal detection apparatus of the present invention preferably further comprises a mask information memory for storing mask information, wherein the vessel noise eliminator eliminates at least one of brightness change information and a characterizing portion from at least one of the differentiated images in the differentiated image memory and the binarized images in the binarized image memory on the basis of the mask information.

The protein crystal detection apparatus of the present invention preferably further comprises a line-thinned image production component for obtaining a line-thinned image constituted by a plurality of lines by subjecting the differentiated image to line thinning after being binarized at a noise extraction threshold, a line-thinned image memory for storing the line-thinned images produced by the line-thinned image production component, a noise recognition processor for detecting lines considered to be noise by individually recognizing the shapes of the plurality of lines included in the line-thinned images in the line-thinned image memory, and a noise eliminator for eliminating at least one of brightness change information and a characterizing portion corresponding to the lines considered to be noise and detected by the noise detection processor, from at least one of the differentiated images in the differentiated image memory and the binarized images in the binarized image memory.

With the protein crystal detection apparatus of the present invention, it is preferable that the noise recognition processor finds the length and number of branches of lines, and any lines whose length is over a predetermined specific value and whose number of branches is less than a predetermined value are considered to be noise.

With the protein crystal detection apparatus of the present invention, it is preferable that the noise recognition processor recognizes the length and linearity of lines, and any lines whose length is over a predetermined specific value and whose linearity is higher than a predetermined value are considered to be noise.

With the protein crystal detection apparatus of the present invention, it is preferable that the noise recognition processor finds the length of lines and the dispersion extent compared to the approximated line of the detected line, and any lines whose length is over a predetermined specific value and whose dispersion extent is less than a predetermined value are considered to be noise.

The protein crystal detection apparatus of the present invention preferably further comprises an observation stage on which is set a crystallization vessel that holds the protein solution to be crystallized, and a camera that captures images by photographing the protein solution in the crystallization vessel set on the observation stage, wherein the images of the protein solution captured by the camera are stored in the observed image memory.

The protein crystal detection apparatus of the present invention also can be used for detecting protein crystals generated by an apparatus for screening protein crystallization conditions. Furthermore, the protein crystal detection apparatus of the present invention can be used for monitoring or detecting protein crystals in a protein crystal manufacturing apparatus. Other than the use of the detection apparatus of the present invention, there are no particular restrictions on this screening apparatus or crystal manufacturing apparatus, and any conventional technique can be utilized.

Next, in the protein crystal detection program of the present invention, it is preferable that the protein crystal detection processing further comprises a vessel noise elimination step of eliminating at least one of brightness change information and a characterizing portion originating in the crystallization vessel that holds the protein solution to be crystallized, from at least one of the differentiated image and the binarized image.

In the protein crystal detection program of the present invention, it is preferable that the noise elimination step involves eliminating at least one of brightness change information and a characterizing portion of a specific region on the basis of given information.

In the protein crystal detection program of the present invention, it is preferable that the protein crystal detecting processing further comprises a line-thinned image production step of obtaining a line-thinned image constituted by a plurality of lines by subjecting the differentiated image to line thinning after being binarized at a noise extraction threshold, a noise recognition step of detecting lines considered to be noise by individually recognizing the shapes of the plurality of lines included in the line-thinned image, and a noise elimination step of eliminating at least one of brightness change information and characterizing portion corresponding to the lines considered to be noise and detected in the noise detection step, from at least one of the differentiated image and the binarized image.

In the protein crystal detection program of the present invention, it is preferable that, in the noise recognition step, the length and number of branches of lines are found, and any lines whose length is over a predetermined specific value and whose number of branches is less than a predetermined value are considered to be noise.

In the protein crystal detection program of the present invention, it is preferable that, in the noise recognition step, the length and linearity of lines are recognized, and any lines whose length is over a predetermined specific value and whose linearity is higher than a predetermined value are considered to be noise.

In the protein crystal detection program of the present invention, it is preferable that, in the noise recognition step, the length of lines and the dispersion extent compared to the approximated line of the detected line are found, and any lines whose length is over a predetermined specific value and whose dispersion extent is less than a predetermined value are considered to be noise.

In the protein crystal detection program of the present invention, it is preferable that the protein crystal detection processing further comprises an observation step observing the protein solution held in a crystallization vessel and capturing the observed image with a camera, and a storage step of storing in an observed image memory the observed image captured by the camera, wherein the differentiation step is performed on images stored in the observed image memory.

An example of an embodiment of the present invention will now be described through reference to the drawings.

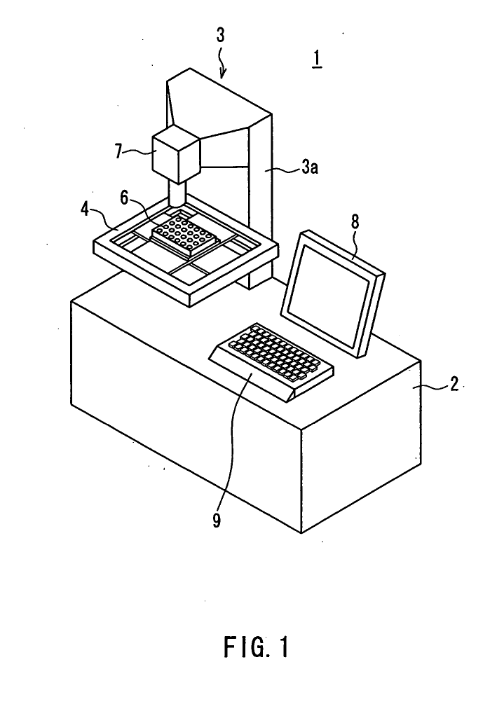

First, an example of the overall structure of the protein crystal detection apparatus will be described through reference to FIG. 1. A protein crystal detection apparatus 1 is a dedicated observation apparatus used in observation for detecting protein crystals produced in a protein solution by vapor diffusion or another such method, for example. In FIG. 1, an observation component 3, a display device 8, and a command input keyboard 9 are provided on a stand 2.

The observation component 3 is configured such that an observation stage 4 is mounted horizontally on a frame 3a, and a camera 7 is disposed above the observation stage 4. A microplate 6 used for crystallization (hereinafter referred to simply as a crystallization plate 6) is set on the observation stage 4. The crystallization plate 6 is a crystallization vessel used for the crystallization of protein in a protein solution. The crystallization plate 6 can be moved in the X, Y, and Z directions by driving an XYZ movement mechanism provided to the observation stage 4.

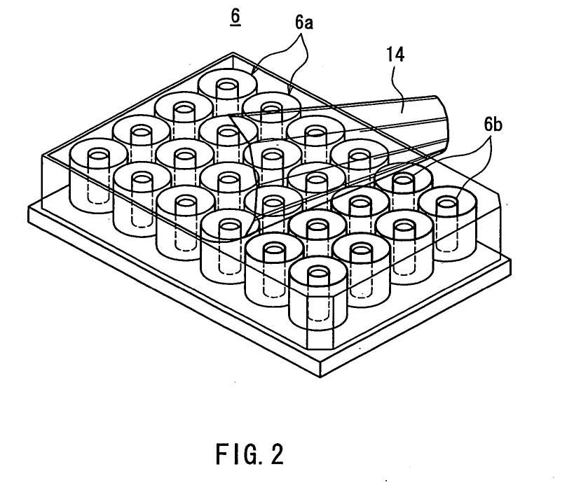

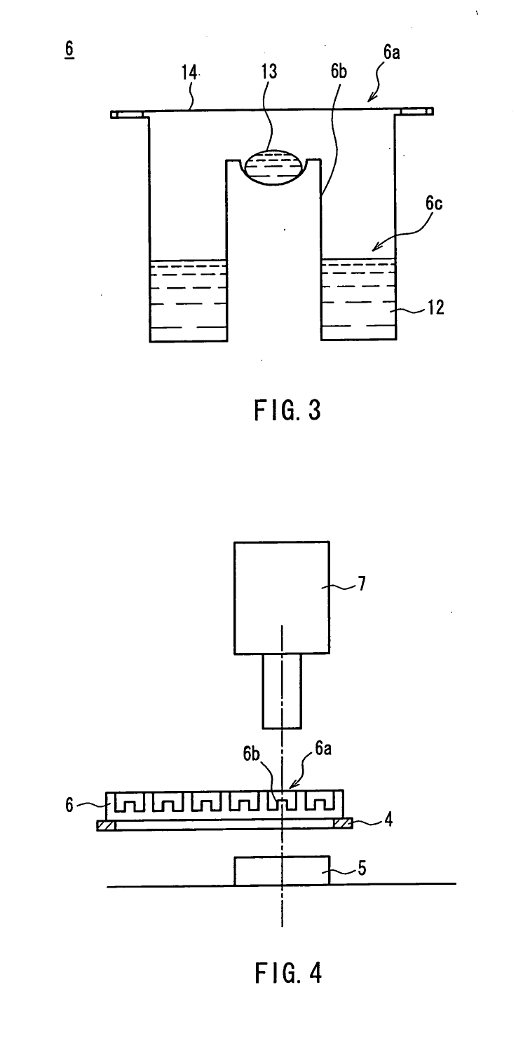

An example of the crystallization plate 6 will be described through reference to FIGS. 2 and 3. As shown in FIG. 2, a plurality of wells 6a are formed in a grid pattern in the crystallization plate 6. The wells 6a are so-called caldera-shaped recesses for holding a liquid, and cylindrical liquid holders 6b are provided in the center of these circular recesses. A protein solution 13 containing the sample to be crystallized, namely, the protein to be crystallized, and a crystallization solution 12 used for crystallization are aliquoted in the wells 6a. There are no particular restrictions on the size of the crystallization plate 6, but one of standardized size can be used, for example. An example of such standards are SBS standards. There are no particular restrictions on the size of the wells 6a, but an example is a diameter of 10 to 20 mm. Neither are there are any particular restrictions on the size of the liquid holders 6b, but an example is a size that is half the diameter of the wells 6a.

FIG. 3 is an example of a cross section of one of the wells 6a holding a sample. Inside the well 6a, a droplet of the protein solution 13 is held resting inside a pocket provided at the top of the liquid holder 6b. The crystallization solution 12 fills a ring-shaped reservoir 6c that surrounds the liquid holder 6b. The well 6a is a liquid container having the liquid holder 6b, with which the protein solution to be crystallized is held from below in a resting state, and the reservoir 6c filled with the crystallization solution 12. As discussed below, upon commencing crystallization, a specific amount of the crystallization solution 12 is taken out of the reservoir 6c and aliquoted and mixed into the protein solution 13 held by the liquid holder 6b, after which a seal 14 is applied to the top surface of each well 6a (see FIG. 2, for example).

The crystallization plate 6 in this state is stored under a specific temperature environment, which causes the solvent component to evaporate from the protein solution 13, and this raises the protein concentration of the protein solution 13 to the supersaturation state and produces protein crystals. At this point, the solvent evaporated from the protein solution 13 and the vapor absorbed by the crystallization solution 12 are held in a state of equilibrium while the evaporation of solvent from the protein solution 13 proceeds gently, with the result being stable crystal production.

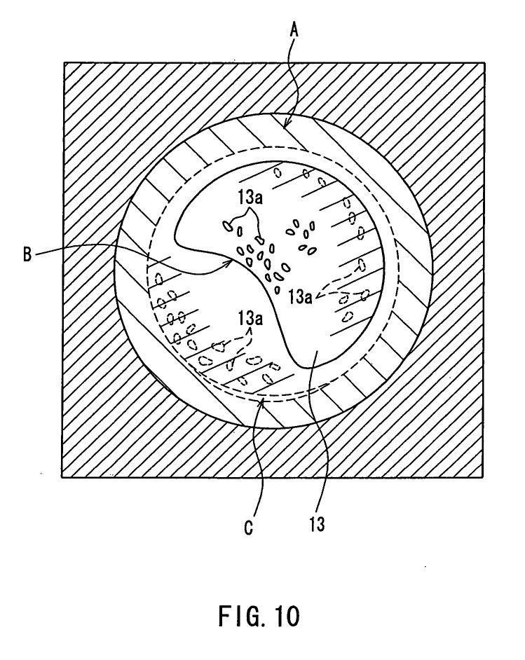

The observation component 3 in FIG. 1 detects whether or not there are protein crystals in the wells 6a, and the extent of any such crystallization, by observing the crystallization plate 6 in the course of this crystal production. Specifically, as shown in FIG. 4, for example, the crystallization plate 6 set on the observation stage 4 is moved underneath the camera 7, and the liquid holder 6b in the well 6a to be observed is positioned within the capturing optical axis of the camera 7. An observed image of the protein solution held in the crystallization plate 6 is captured by photographing an image of the crystallization plate 6 with the camera 7 while illuminating the crystallization plate 6 from below with an illumination apparatus 5 (see FIG. 10, for example).

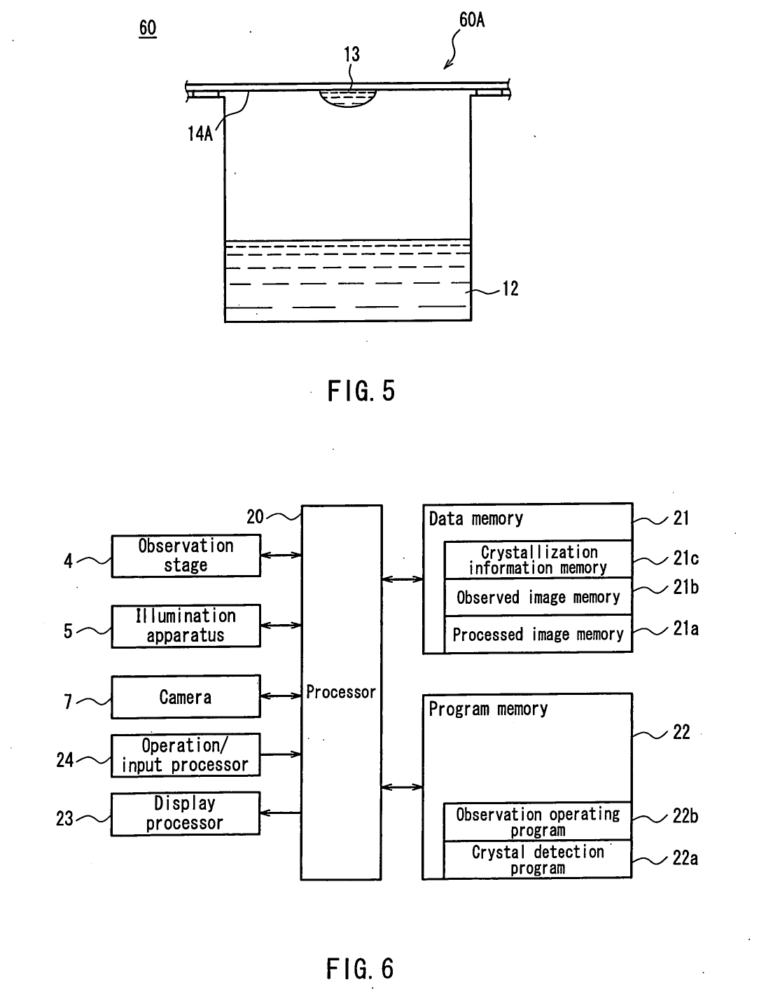

FIGS. 2 and 3 are examples of the crystallization plate 6 used in vapor diffusion that involves a sitting drop method, in which the protein solution 13 is supported from below in a resting state in the course of crystal production, but in addition to this, a well-shaped crystallization plate 60 such as that shown in FIG. 5 may be used. This crystallization plate 60 is used with a hanging drop method, in which a droplet of the protein solution 13 is held in a suspended state.

In the example shown in FIG. 5, a well 60A has only a reservoir filled with the crystallization solution 12, and no liquid holder is formed, so the droplet of the protein solution 13 to be crystallized (in which a specific amount of the crystallization solution 12 has been mixed) is supported in a suspended state from the back side of a glass plate 14A that blocks off the well 60A. Specifically, the glass plate 14A onto which the protein solution 13 has been dropped is turned over and affixed so as to block off the well 60A into which the crystallization solution 12 has been aliquoted, thereby sealing the well 60A.

Next, an example of the constitution of the control system of the protein crystal detection apparatus will be described through reference to FIG. 6. In FIG. 6, a processor 20 executes various processing programs stored in a program memory 22 on the basis of various data stored in a data memory 21, thereby performing various operational and processing functions (discussed below). Here, a crystal detection program 22a and an observation operating program 22b are stored in the program memory 22, and these programs are executed to perform observation of the protein solution and detection processing of the protein crystals in the protein solution (discussed below).

The data memory 21 is equipped with a processed image memory 21a, an observed image memory 21b, and a crystallization information memory 21c. The processed image memory 21a stores processed images that have undergone various kinds of processing in the protein crystal detection processing. The observed image memory 21b stores observed images of the protein solution 13 taken with the camera 7 (see FIG. 10, for example).

In protein crystal detection processing (discussed below), the object of the processing is the observed images stored in the observed image memory 21b. The crystallization information memory 21c stores crystallization information, that is, image data about observed images in which crystallization has been detected in protein crystal detection processing, information identifying plate wells from which observed images have been obtained, observation time at which these plates were observed, and other such information.

A display processor 23, a operation/input processor 24, the camera 7, the illumination apparatus 5, and the observation stage 4 are connected to the processor 20. In an observation operation, the processor 20 controls the observation stage 4, the illumination apparatus 5, and the camera 7, thereby moving the crystallization plate 6 supported on the observation stage 4, illuminating the crystallization plate 6 with the illumination apparatus 5, and capturing images of the protein solution with the camera 7. The display processor 23 displays observed images captured by the camera 7 and various kinds of processed images, and also displays instruction images for data input and so forth, all on the display device 8. The operation/input processor 24 is used to issue operation commands or input data to the processor 20 by operating the keyboard 9 or another such input device.

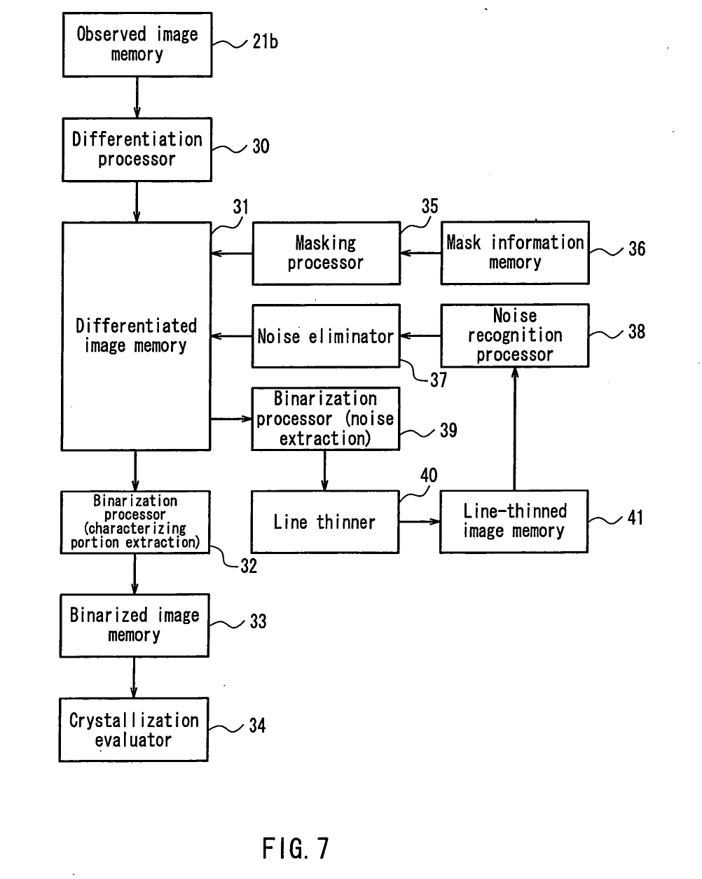

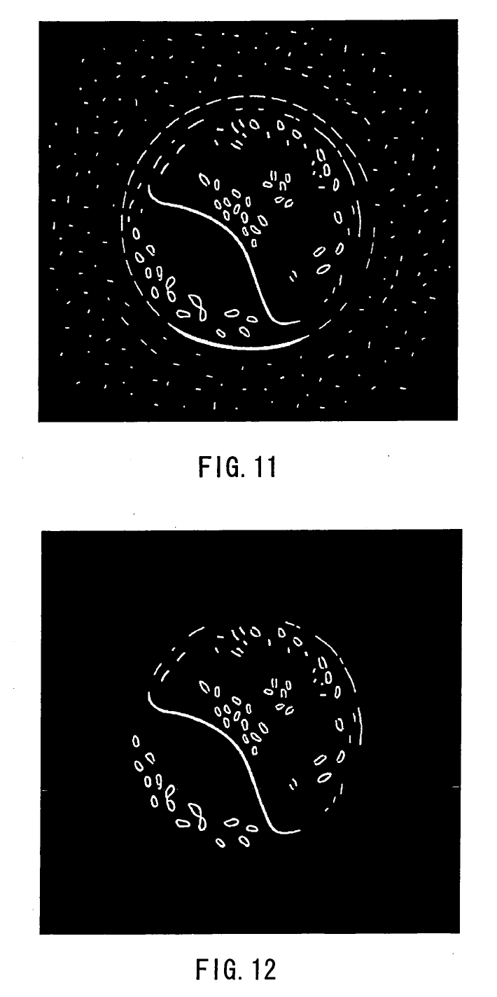

Next, an example of the protein crystal detection processing function will be described through reference to the function block diagram in FIG. 7. In FIG. 7, a differentiation processor 30 differentiates observed images stored in the observed image memory 21b (see FIG. 10, for example), that is, images of the protein solution captured by the camera 7, and thereby produces differentiated images constituted by brightness change information indicating how much the brightness has changed (see FIG. 11, for example).

The term “brightness change information” as used here refers to the rate of change of brightness in the pixels that make up an observed image, given as a numerical value (brightness change value).

Within an observed image, there is little change in brightness in those regions where no observation object is present, such as background regions or portions that transmit illumination light, but there is considerable change in brightness in those portions corresponding to silhouettes of protein crystals (the object of detection). Therefore, in the above-mentioned differentiated images, silhouettes of observation objects captured in images are extracted as portions of large brightness change.

However, in these differentiated images, not the protein crystals that are the target of detection, but also sediment and other such solid foreign matter other than crystals present in the protein solution, the shape of the liquid holders 6b in the wells 6a, silhouettes of droplets, and so forth are similarly extracted as portions of large brightness change, so noise elimination processing (discussed below) is performed to eliminate as noise any portions of large brightness change other than the detection targets.

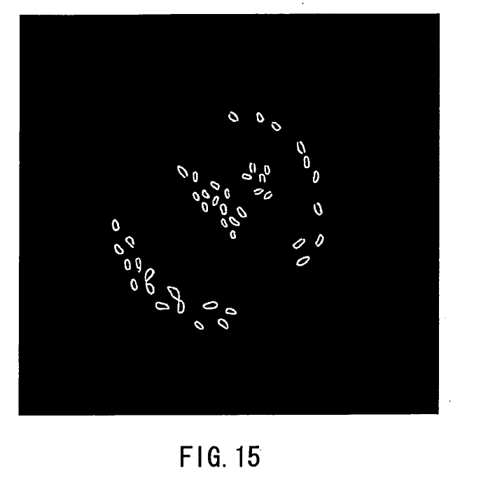

A differentiated image memory 31 in FIG. 7 stores differentiated images produced by the differentiation processor 30. A binarization processor 32 binarizes the differentiated images in the differentiated image memory 31 at a threshold for extracting a characterizing portion, thereby giving binarized images in which the portions of the brightness change information exhibiting a large change in brightness are left behind as the characterizing portion (see FIG. 15, for example). A binarized image memory 33 stores binarized images obtained from the binarization processor 32. A crystallization evaluator 34 determines whether or not there are protein crystals from the characterizing portions of the binarized images stored in the binarized image memory 33. There are no particular restrictions on how the threshold for extracting the characterizing portion is set, but an example is a method in which the characterizing portion of the crystals to be detected is suitably set to an extractable value using a crystal sample, image sample, or the like prepared in advance.

Here, the differentiated images to be binarized by the binarization processor 32 are subjected to masking and noise elimination processing as described below. In order to recognize the noise that is to be eliminated, binarization and line thinning for noise extraction are executed prior to the noise elimination processing.

First, the masking processing will be described. A mask information memory 36 stores mask information, namely, brightness change information that appears in the differentiated images and can be traced to the shape of the crystallization plate 6 (the shape of the liquid holders 6b; see FIGS. 2 and 3, for example), which is the crystallization vessel that holds the protein solution, within the observed images, and more specifically, mask information (here, the diameter of the pockets provided to the liquid holders 6b) for eliminating from the differentiated images any vessel noise whose appearance can be traced to the shape of the crystallization plate 6 (the vessel), out of the noise contained in the observed images of the protein solution that are supposed to be observed. A masking processor 35 performs masking processing that eliminates vessel noise from the differentiated images on the basis of the mask information stored in the mask information memory 36. The result is that differentiated images from which vessel noise has been eliminated are obtained (see FIG. 12, for example).

Noise elimination processing now will be described. The noise elimination processing performed here is aimed at noise that cannot be foreseen, such as noise that cannot be eliminated by the above-mentioned masking processing, an example of which is the silhouettes of droplets of protein solution appearing in the pockets at the top of the liquid holders 6b. Accordingly, a noise recognition processor 38 recognizes which portion of the information contained in a differentiated image is noise, and a noise eliminator 37 eliminates noise from the differentiated images on the basis of this noise recognition result. This gives a differentiated image from which noise has been eliminated (see FIG. 14, for example).

The means for recognizing noise is to identify, on the basis of the shape characteristics of the lines in a line-thinned image produced by the line thinning of a differentiated image, whether these lines are shape lines indicating a shape such as the outline of a figure that indicates a protein crystal, or are lines that are clearly different from those of a figure indicating a protein crystal, such as the silhouette of a droplet.

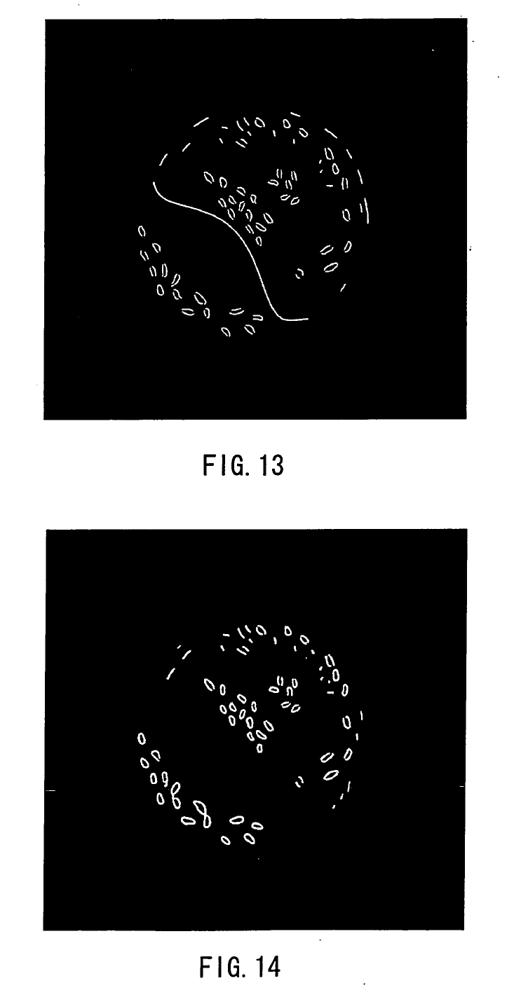

Specifically, in the noise recognition given here, first a differentiated image stored in the differentiated image memory 31 is binarized at a noise extraction threshold by a binarization processor 39. A line thinner 40 subjects the binarized images resulting from the binarization processing to line thinning. Specifically, the line elements that make up a binarized image are replaced with thin lines with the width of one pixel. This produces a line-thinned image constituted by a plurality of lines in which the range indicating a portion of large brightness change in a differentiated image has undergone line thinning (see FIG. 13, for example). There are no particular restrictions on how to set the noise extraction threshold, but an example is a method in which it is set to a suitable value at which a distinction can be made between the brightness change information of the protein crystals that are the target of detection and the brightness change information of sediment, droplets, and the like other than these crystals, using a crystal sample, image sample, or the like prepared in advance.

Therefore, the binarization processor 39 and the line thinner 40 constitute a line-thinned image production component with which a line-thinned image constituted by a plurality of lines is obtained by binarizing a differentiated image at a noise extraction threshold and then performing line thinning. A line-thinned image memory 41 stores line-thinned images produced by the line-thinned image production component.

The noise recognition processor 38 detects lines that are considered to be noise by individually recognizing the shapes of the plurality of lines included in the line-thinned images in the line-thinned image memory 41. The noise eliminator 37 eliminates from the differentiated images in the differentiated image memory 31 any brightness change information corresponding to lines detected as noise by the noise recognition processor 38 among the differentiated images stored in the differentiated image memory 31.

Here, the noise recognition processor 38 uses a plurality of algorithms in the detection of lines considered to be noise, as discussed below. Specifically, it makes use of three different algorithms as necessary, either separately or together: a first algorithm that finds the length and number of branches of lines, and considers to be noise any lines whose length is over a predetermined specific value and whose number of branches is small, a second algorithm that recognizes the length and linearity of lines, and considers to be noise any lines whose length is over a predetermined specific value and whose linearity is high, and a third algorithm that finds the length of lines and the dispersion extent compared to the approximated line of the detected line, and considers to be noise any lines whose length is over a predetermined specific value and whose dispersion extent is small.

In the above examples, the differentiated images stored in the differentiated image memory 31 were the object of processing in the vessel noise elimination processing by the masking processor 35 and the noise elimination processing by the noise eliminator 37, but the constitution may instead be such that the binarized images stored in the binarized image memory 33 are the object, and are subjected to vessel noise elimination processing by the masking processor 35 and noise elimination processing by the noise eliminator 37.

The protein crystal detection method now will be described. First, an observation operation is executed when the processor 20 in FIG. 6 executes an observation operating program 22b. Specifically, the camera 7 captures image of the protein solution 13 supported by the liquid holders 6b of the wells 6a in the crystallization plate 6 (observation step), and the captured images are stored in the observed image memory 21b (storage step). As a result, observed images of the processing objects are stored in the observed image memory 21b.

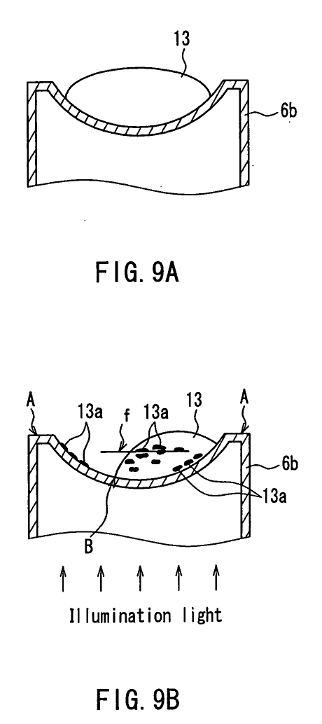

The change in the protein solution 13 over time (the observation object) now will be described through reference to FIG. 9. As discussed above, at the start of crystallization, droplets of the protein solution 13 to which the crystallization solution 12 has been added in a specific proportion are placed in the liquid holders 6b of the wells 6a (see FIG. 3). FIG. 9A shows an example of a cross section of a liquid holder 6b at the outset of crystallization. At this stage, the droplet of protein solution 13 substantially fills the pocket of the liquid holder 6b, and no protein crystals are as yet seen inside the droplet.

If the crystal growth conditions here are favorable, that is, if the composition and concentration of the crystallization solution 12 and the storage temperature are favorable, crystallization will occur within the protein solution 13 as time passes. FIG. 9B shows an example of a cross section of a liquid holder 6b in a state in which crystallization has progressed somewhat. In this state, the solvent component in the protein solution 13 gradually evaporates, and as the droplet inside the pocket of the liquid holder 6b shrinks as a result, the silhouette of the droplet (indicated by arrow B) moves further toward the inside of the pocket than in the state shown in FIG. 9A. This shrinkage of the droplet leaves behind protein crystals 13a, which is the result of the crystallization of the protein contained in the droplet, on the exposed bottom of the pocket. Crystallization proceeds all the way into the interior of the droplet, producing the protein crystals 13a.

FIG. 10 shows an example of an observed image in the state shown in FIG. 9B, and includes an image taken from above of the droplet whose volume shrunk after evaporation of the solvent in the pocket of the liquid holder 6b has progressed a certain amount. In this observed image, as shown in FIG. 10, an annular portion A (indicated by light hatching) indicating the top of the liquid holder 6b appears at relatively high brightness in the low-brightness background image (indicated by heavy hatching).

The droplet of protein solution 13 appears as an image in which the brightness varies with the portion in the pocket on the inside of this annular portion A. The portion where no droplet is present on the inside of the annular portion A is a high-brightness portion because it transmits the illumination light from below (see FIG. 9B), and the silhouette B of the droplet of protein solution 13 appears distinctly in the observed image because of its contrast with this high-brightness portion. A linear high-brightness portion C appears locally around the inner periphery of the annular portion A.

The protein crystals 13a that are the detection object are present in the region on the inside of the annular portion A. Specifically, of the protein crystals 13a present in the interior of the droplet of protein solution 13, those protein crystals 13a that are present near the focal level f of the camera 7 (see FIG. 9B) appear distinctly and in high focus in the observed image of FIG. 10, and the silhouettes of the protein crystals 13a that are located farther away from the focal level f appear blurrier (less in focus). Similarly, the protein crystals 13a outside the droplet, that are present in a form remaining on the surface of the pocket, are less in focus and have a blurrier silhouette. In addition to these, there is in the observed image sediment and other such solid foreign matter that did not undergo crystallization.

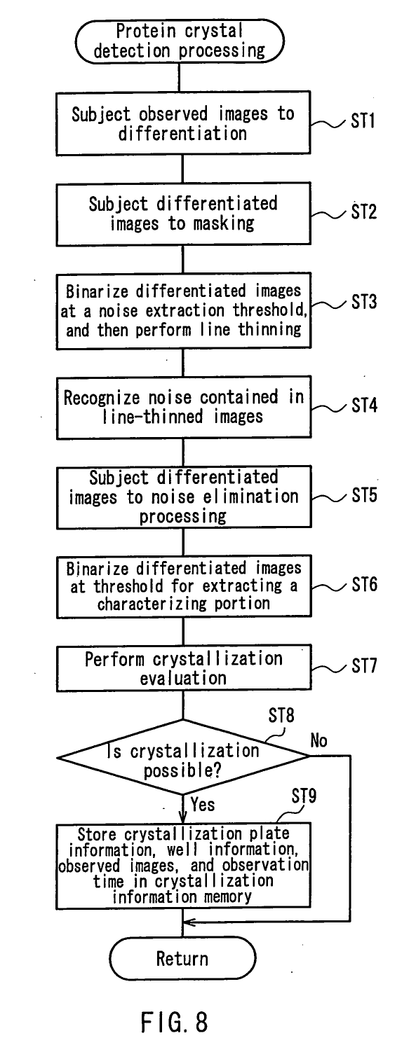

In the detection of protein crystals, it is difficult accurately to distinguish these figures that appear in an observed image by visual observation, and protein crystals (the object of detection) cannot be efficiently detected at a high reliability, so with the protein crystal detection method of this embodiment, the processor 20 executes the crystal detection program 22a stored in the program memory 22 of FIG. 6, the result being that the observed image is subjected to protein crystal detection processing as shown in the flowchart of FIG. 8, and protein crystals are identified automatically.

In the protein crystal detection processing in FIG. 8, first the observed images are differentiated (ST1). Specifically, differentiated images composed of brightness change information indicating how much the brightness has changed are produced by differentiating the observed images of the protein solution stored ahead of time in the observed image memory 21b (differentiation step). As a result, as shown in FIG. 11, portions with a large change in brightness, such as the droplet silhouette B and the high-brightness portion C on the inner periphery of the annular portion A, or shape lines indicating the outer shape of the protein crystals 13a appearing in the observed image of FIG. 10, appear at a high brightness level. A differentiated image is actually a multi-valued image in which the pixels appear at a brightness corresponding to the brightness change value, but in FIG. 11 this is expressed by a black and white binary image for the sake of simplicity in the drawing.

Next, these differentiated images are subjected to masking (ST2). Here, any brightness change information attributable to the liquid holders 6b of the crystallization plate 6 supporting the protein solution 13 is eliminated from the differentiated image (vessel noise elimination step). This vessel noise elimination is carried out by eliminating brightness change information of a specific region where there is no possibility of the protein solution 13 being supported, on the basis of mask information given ahead of time as the shape of the liquid holders 6b of the crystallization plate 6.

Of the brightness change information in the differentiated image shown in FIG. 11, the portion with a large brightness change present outside the range where the protein solution 13 is supported, that is, the portion where the brightness change is large and which includes the inner peripheral part of the annular portion A shown in FIG. 10 and appears farther to the outside than these, is eliminated as noise. As a result, as shown in FIG. 12, a differentiated image (masked) is obtained that includes only the region where there is a possibility of protein crystals being present.

The differentiated image that thus has been masked is binarized at a noise extraction threshold, and then subjected to line thinning (ST3). FIG. 13 shows a line-thinned image obtained by this line thinning. The high-brightness portions in the differentiated image, that is, the droplet silhouette, the shape lines of the protein crystals 13a, and so forth, have all been replaced with lines whose width is that of one pixel. Noise extraction is performed on the basis of the line-thinned image thus produced. Specifically, noise included in the line-thinned image is recognized (ST4).

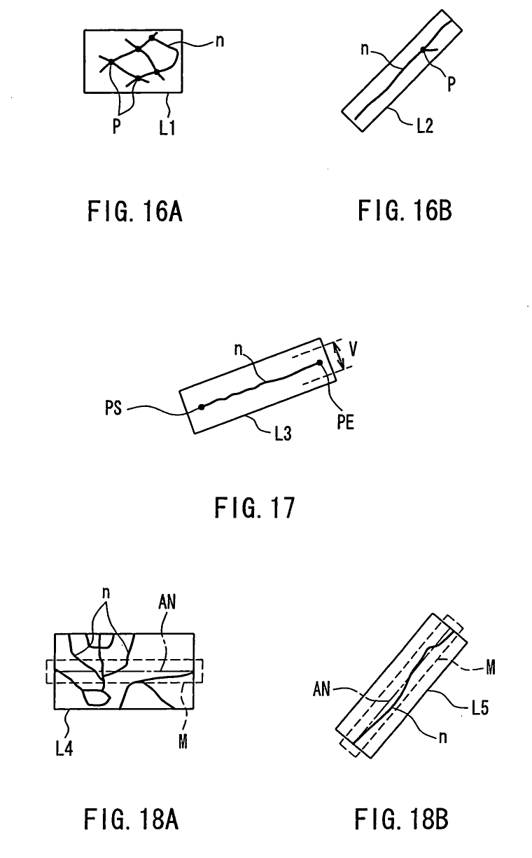

An example of a noise recognition method will now be described through reference to FIGS. 16 to 18. All the examples shown in FIGS. 16 to 18 are of detecting as noise anything that has a certain amount of continuous length, such as a line corresponding to the above-mentioned silhouette B, and does not match the characteristic shape of a protein crystal. Here, a plurality of lines in the line-thinned image are labeled collectively, and of the individual labels, for those in which the line length exceeds a specific value, it is recognized with an algorithm (described below) whether or not they correspond to noise. In this case, since images that have been line-thinned are labeled, the label surface area itself indicates the length of the lines in each label.

FIGS. 16A and 16B illustrate the first algorithm used in this noise recognition. First, the branching points where the lines that make up a label branch off are found for labels whose line length exceeds a specific value, and whether or not something is noise is determined on the basis of the number of branching points found. For instance, with the example of label L1 shown in FIG. 16A, the length of line n is over the specific value, but there are many branching points P, so this is not considered to be noise.

In contrast, in the example of label L2 shown in FIG. 16B, the length of line n is over the specific value, and furthermore there is only one branching point P, and the cutoff at which it is determined that “there are few branching points” has not been exceeded, so this is considered to be noise. Specifically, with the example of the first algorithm, in the noise recognition step, the length of the lines and the number of branches are found, and lines whose length exceeds the specific value and have few branches are considered to be noise. Another method that may be employed for determining whether or not something is noise on the basis of the number of branching points is to determine a line to have few branches if the quotient of dividing the length of a line by the number of branches is not over a specific length.

FIG. 17 illustrates the second algorithm. Here, whether or not something is noise is determined by evaluating the linearity of the lines that make up a label, for those labels whose line length exceeds the specific value. For instance, with the label L3 shown in FIG. 17, a search is performed for lines n that make up the label, linearity is deemed high when a line n for which a search was started at a search starting point PS is within a range of permissible search width V preset at a search end point PE, and this line n is concluded to be noise. Specifically, in the example of the second algorithm, in the noise recognition step, the length and linearity of a line are recognized, and a line that has high linearity and whose length is over a specific value is considered to be noise.

FIG. 18 illustrate the third algorithm. Here, whether or not something is noise is determined by finding the dispersion extent of the plurality of lines that make up a label, with respect to an approximated line. For instance, with the label L4 shown in FIG. 18A, an approximated line AN is found that approximates all of the lines n that make up the label. Then the dispersion extent of the lines n with respect to the approximated line AN within a specific box M that sandwiches the approximated line AN is found. In this case, since the plurality of lines n are intertwined, the dispersion extent is large, and this is not considered to be noise.

In contrast, in the example of the label L5 shown in FIG. 18B, the line n follows the approximated line AN fairly closely, and the dispersion extent with respect to the approximated line AN is small, so this is considered to be noise. Specifically, in the example of the third algorithm, in the noise recognition step, the length of the lines and the dispersion extent of these lines with respect to an approximated line are found, and the lines are considered to be noise if their length is over a specific value and their dispersion extent is small. There are no particular restrictions on how the various parameters are set in this noise recognition method, but an example is a method in which the parameters are suitably set to values at which the noise to be eliminated will be detected, using noise and crystal samples or the like prepared in advance.

If noise has been recognized in this manner, noise elimination processing is performed to eliminate any lines that have been detected as noise from the differentiated images that have been binarized (ST5). Specifically, in ST3, ST4, and ST5, a line-thinned image constituted by a plurality of lines is obtained by binarizing and then line-thinning a differentiated image at a noise extraction threshold (line-thinned image production step).

The shapes of the plurality of lines included in the line-thinned image thus produced then are individually recognized to detect any lines considered to be noise (noise recognition step), and brightness change information corresponding to lines detected as noise in the noise recognition step is eliminated from the differentiated image (noise elimination step). FIG. 14 shows a differentiated image from which noise has thus been eliminated (noise eliminated).

After this, a characterizing portion with a high probability of corresponding to protein crystals is extracted. First, a differentiated image (noise eliminated) is binarized at a threshold for extracting a characterizing portion (ST6). Specifically, when the differentiated image is binarized at a threshold for extracting a characterizing portion, a binarized image in which brightness change information indicating a large change in brightness is left behind as a characterizing portion (binarization step). The threshold used here is set to a higher value than the noise extraction threshold discussed above.

In the binarization step, the characterizing portions are labeled, just the labels whose surface area is larger than a specific value are left, and everything else is erased. As a result, characterizing portions with a low probability of being protein crystals are considered to be noise and are eliminated. FIG. 15 shows a binarized image obtained in this way. In this image, only the characterizing portions with a high probability of being protein crystals appear. The image thus obtained is stored in the binarized image memory 33.

The vessel noise elimination step and noise elimination step discussed above may be performed on the binarized images stored in the binarized image memory 33. In this case, of the characterizing portions left in the binarized image, those corresponding to lines detected as noise are eliminated from the binarized images.

After this, the binarized images thus obtained are subjected to crystallization evaluation (ST7). For instance, when there are few characterizing portions in a binarized image, it is determined that there is no possibility for crystallization to occur or to progress, and when there are many characterizing portions, it is determined that there is a possibility for crystallization to occur or to progress. Whether there are many or few characterizing portions is decided by comparing the total surface area equivalent to the characterizing portions with a specific value. There are no particular restrictions on this specific value, but for example, it can be suitably set using the surface area corresponding to the characterizing portions calculated using a crystal sample, image sample, or the like prepared in advance. Specifically, whether or not there are protein crystals and/or whether or not crystallization has proceeded is determined from the characterizing portions of the binarized images (crystallization evaluation step).

If it has been determined here that there is no possibility for crystallization, the processing is concluded. If it has been determined in ST8 that there is a possibility for crystallization, then crystallization information, such as the observation time, observed images, well information, or crystallization plate information indicating the crystallization plate 6 in which observed images that were the object of the processing in question were obtained, is stored in the crystallization information memory 21c (ST9), and protein detection processing aimed at these observed images is concluded.

As described above, in protein crystal detection in this embodiment, an image of a protein solution is differentiated so that the portion corresponding to the silhouette of the protein crystals to be detected will first be extracted as a portion of large brightness change. This portion of large brightness change is then divided at a threshold for extracting a characterizing portion, to find a binarized image in which the portion indicating a large change in brightness is left behind as a characterizing portion. Whether or not there are protein crystals is then determined from the characterizing portions of this binarized image. As a result, it is possible to eradicate the inconsistency in the reliability of detection results and the efficiency of crystal detection work due to differences in individual skill levels, and protein crystals can be efficiently detected as a high reliability.

In this embodiment, processing was performed with the masking processor 35 and the noise eliminator 37 so that no noise would be included in the characterizing portions of the binarized images, but when observed images containing almost no noise resulting from the shape of the vessel, illumination conditions, and so forth are obtained, the processing with the masking processor 35 and the noise eliminator 37 can be omitted.

### INDUSTRIAL APPLICABILITY

In the past, the detection of protein crystals relied exclusively on visual observation by observers, but with the present invention, the detection of protein crystals can be automated, and can be performed efficiently and with high reliability. Also, the protein crystal detection method of the present invention can be applied to the screening of protein crystallization conditions by vapor diffusion or another such method, and the protein crystal detection apparatus of the present invention can be used as a protein crystal manufacturing apparatus or an apparatus for screening protein crystallization conditions by vapor diffusion or another such method, for example.

1. A protein crystal detection method for detecting protein crystals, comprising:
a differentiation step of observing a protein solution and differentiating the observed image, thereby producing a differentiated image constituted by brightness change information indicating how much brightness has changed;
a binarization step of binarizing the differentiated image at a threshold for extracting a characterizing portion, thereby obtaining a binarized image in which the portion of the brightness change information exhibiting a large change in brightness is left behind as the characterizing portion; and
a crystallization evaluation step of determining from the characterizing portion of the binarized image whether or not there are protein crystals and/or whether or not crystallization has proceeded.

2. The method for detecting protein crystals according to claim 1, further comprising a vessel noise elimination step of eliminating at least one of brightness change information and a characterizing portion originating in the crystallization vessel that holds the protein solution to be crystallized, from at least one of the differentiated image and the binarized image.

3. The method for detecting protein crystals according to claim 2, wherein the vessel noise elimination step involves eliminating at least one of brightness change information and a characterizing portion of a specific region on the basis of given information.

4. The method for detecting protein crystals according to claim 1, further comprising a line-thinned image production step of obtaining a line-thinned image constituted by a plurality of lines by subjecting the differentiated image to line thinning after being binarized at a noise extraction threshold;
a noise recognition step of detecting lines considered to be noise by individually recognizing the shapes of the plurality of lines included in the line-thinned image; and
a noise elimination step of eliminating at least one of brightness change information and a characterizing portion corresponding to the lines considered to be noise and detected in the noise recognition step, from at least one of the differentiated image and the binarized image.

5. The method for detecting protein crystals according to claim 4, wherein, in the noise recognition step, the length and number of branches of lines are found, and any lines whose length is over a predetermined specific value and whose number of branches is less than a predetermined value are considered to be noise.

6. The method for detecting protein crystals according to claim 4, wherein, in the noise recognition step, the length and linearity of lines are recognized, and any lines whose length is over a predetermined specific value and whose linearity is higher than a predetermined value are considered to be noise.

7. The method for detecting protein crystals according to claim 4, wherein, in the noise recognition step, the length of lines and the dispersion extent compared to the approximated line of the detected line are found, and any lines whose length is over a predetermined specific value and whose dispersion extent is less than a predetermined value are considered to be noise.

8. The method for detecting protein crystals according to claim 1, further comprising an observation step of capturing with a camera an image of a protein solution held in a crystallization vessel; and
a storage step of storing in an observed image memory the observed image captured by the camera,
wherein the differentiation step is performed on images stored in the observed image memory.

9. A protein crystal detection apparatus for detecting protein crystals, comprising:
an observed image memory for storing observed images obtained by observing a protein solution;
a differentiation processor for differentiating an observed image in the observed image memory and thereby producing a differentiated image constituted by brightness change information indicating how much brightness has changed;
a binarization processor for binarizing the differentiated image produced by the differentiation processor at a threshold for extracting a characterizing portion, thereby obtaining a binarized image in which the portion of the brightness change information exhibiting a large change in brightness is left behind as the characterizing portion;
a binarized image memory for storing binarized images obtained from the binarization processor; and
a crystallization evaluator for determining from the characterizing portion of the binarized images in the binarized image memory whether or not there are protein crystals and/or whether or not crystallization has proceeded.

10. The protein crystal detection apparatus according to claim 9, further comprising a vessel noise eliminator for eliminating at least one of brightness change information and a characterizing portion originating in the crystallization vessel that holds the protein solution to be crystallized, from at least one of the differentiated images in the differentiated image memory and the binarized images in the binarized image memory.

11. The protein crystal detection apparatus according to claim 10, further comprising a mask information memory for storing mask information, wherein the vessel noise eliminator eliminates at least one of brightness change information and a characterizing portion from at least one of the differentiated images in the differentiated image memory and the binarized images in the binarized image memory on the basis of the mask information.

12. The protein crystal detection apparatus according to claim 9, further comprising a line-thinned image production component for obtaining a line-thinned image constituted by a plurality of lines by subjecting the differentiated image to line thinning after being binarized at a noise extraction threshold;
a line-thinned image memory for storing the line-thinned images produced by the line-thinned image production component;
a noise recognition processor for detecting lines considered to be noise by individually recognizing the shapes of the plurality of lines included in the line-thinned images in the line-thinned image memory; and
a noise eliminator for eliminating at least one of brightness change information and a characterizing portion corresponding to the lines considered to be noise and detected by the noise detection recognition processor, from at least one of the differentiated images in the differentiated image memory and the binarized images in the binarized image memory.

13. The protein crystal detection apparatus according to claim 12, wherein the noise recognition processor finds the length and number of branches of lines, and any lines whose length is over a predetermined specific value and whose number of branches is less than a predetermined value are considered to be noise.

14. The protein crystal detection apparatus according to claim 12, wherein the noise recognition processor recognizes the length and linearity of lines, and any lines whose length is over a predetermined specific value and whose linearity is higher than a predetermined value are considered to be noise.

15. The protein crystal detection apparatus according to claim 12, wherein the noise recognition processor finds the length of lines and the dispersion extent compared to the approximated line of the detected line, and any lines whose length is over a predetermined specific value and whose dispersion extent is less than a predetermined value are considered to be noise.

16. The protein crystal detection apparatus according to claim 9, further comprising an observation stage on which is set a crystallization vessel that holds the protein solution to be crystallized; and
a camera that captures images by photographing the protein solution in the crystallization vessel set on the observation stage,
wherein the images of the protein solution captured by the camera are stored in the observed image memory.

17. A program storage medium storing a protein crystal detection program for executing on a computer processing for detecting protein crystals produced in a protein solution, comprising:
a differentiation step of observing a protein solution and differentiating the observed image, thereby producing a differentiated image constituted by brightness change information indicating how much brightness has changed;
a binarization step of binarizing the differentiated image at a threshold for extracting a characterizing portion, thereby obtaining a binarized image in which the portion of the brightness change information exhibiting a large change in brightness is left behind as the characterizing portion; and
a crystallization evaluation step of determining from the characterizing portion of the binarized image whether or not there are protein crystals and/or whether or not crystallization has proceeded.

18. The program storage medium storing a protein crystal detection program according to claim 17, wherein the protein crystal detection processing further comprises a vessel noise elimination step of eliminating at least one of brightness change information and a characterizing portion originating in the crystallization vessel that holds the protein solution to be crystallized, from at least one of the differentiated image and the binarized image.

19. The program storage medium storing a protein crystal detection program according to claim 18, wherein the vessel noise elimination step involves eliminating at least one of brightness change information and a characterizing portion of a specific region on the basis of given information.

20. The program storage medium storing a protein crystal detection program according to claim 17, wherein the protein crystal detecting processing further comprises a line-thinned image production step of obtaining a line-thinned image constituted by a plurality of lines by subjecting the differentiated image to line thinning after being binarized at a noise extraction threshold;
a noise recognition step of detecting lines considered to be noise by individually recognizing the shapes of the plurality of lines included in the line-thinned image; and
a noise elimination step of eliminating at least one of brightness change information and a characterizing portion corresponding to the lines considered to be noise and detected in the noise recognition step, from at least one of the differentiated image and the binarized image.

21. The program storage medium storing a protein crystal detection program according to claim 20, wherein, in the noise recognition step, the length and number of branches of lines are found, and any lines whose length is over a predetermined specific value and whose number of branches is less than a predetermined value are considered to be noise.

22. The program storage medium storing a protein crystal detection program according to claim 20, wherein, in the noise recognition step, the length and linearity of lines are recognized, and any lines whose length is over a predetermined specific value and whose linearity is higher than a predetermined value are considered to be noise.

23. The program storage medium storing a protein crystal detection program according to claim 20, wherein, in the noise recognition step, the length of lines and the dispersion extent compared to the approximated line of the detected line are found, and any lines whose length is over a predetermined specific value and whose dispersion extent is less than a predetermined value are considered to be noise.

24. The program storage medium storing a protein crystal detection program according to claim 20, wherein the protein crystal detection processing further comprises an observation step observing the protein solution held in a crystallization vessel and capturing the observed image with a camera; and
a storage step of storing in an observed image memory the observed image captured by the camera,
wherein the differentiation step is performed on images stored in the observed image memory.
